(19) United States
(12) Patent Application Publication
(10) Pub. No.: US 2023/0355161 A1
(43) Pub. Date: Nov. 9, 2023

(54) Neural Interface

(71) Applicant: Imperial College Innovations Limited, London (GB)

(72) Inventors: Dario FARINA, Birmingham (GB); Mario BRÄCKLEIN, London (GB); Deren Yusuf BARSAKCIOGLU, London (GB); Jaime IBANEZ PEREDA, London (GB)

(73) Assignee: Imperial College Innovations Limited, London (GB)

(21) Appl. No.: 18/044,892

(22) PCT Filed: Sep. 14, 2021
(86) PCT No.: PCT/GB2021/052375

§ 371 Date: Mar. 10, 2023

(30) Foreign Application Priority Data
Sep. 17, 2020 (GB) GB2014671.8

(51) Int. Cl. A61B 5/397; G06F 3/01

(52) U.S. Cl. CPC A61B 5/397; G06F 3/011

(57) ABSTRACT

An apparatus, computer program and method is described comprising: separating neuron discharge patterns obtained from electromyography signals into one or more frequency band signals based on bandpass filter configuration parameters and converting one or more of said one or more frequency band signals into a one or more control signals, wherein said one or more control signals include one or more first control signals, wherein the or each first control signal is an independent augmented control signal.

### FIELD

The present specification relates to neural interfaces, for example for use as part of a human machine interface (HMI).

### BACKGROUND

Neural interfaces and human-machine interfaces (HMIs) offer significant potential benefits to people who suffer from neuromuscular disorders, paralysis and amputations through advancement of scientific knowledge and development of rehabilitation devices. HMIs can also offer diagnostic tools for the clinical sector. Moreover, with a reliable electrical output signal, HMIs could enable a wide variety of systems to be controlled.

Many neural interface paradigms have been proposed. However, there remains a need for alternative and improved neural interfaces and human-machine interfaces.

### SUMMARY OF THE INVENTION

In an embodiment, there is provided an apparatus (e.g. a human-machine interface) comprising: a filter module for separating neuron discharge patterns obtained from electromyography signals into one or more frequency band signals based on bandpass filter configuration parameters; and an output module for converting one or more of said one or more frequency band signals into a one or more control signals, wherein said one or more control signals include one or more first control signals, wherein the or each first control signal is an independent augmented control signal. The first control signal may be a computer readable signal and may, for example, be integrated into a virtual reality or an augmented reality application or may be provided to control an apparatus or a software application.

The one or more control signals may further comprise one or more second control signals, wherein the or each second control signal is independent of said first control signal. The second control signal(s) may be muscle control signal(s) or some other control signal associated with the natural function of the muscle (e.g. a natural force control signal). The or each second control signal may be provided to one or more external electrical/electro-mechanical apparatus (e.g. a prosthetic limb). Alternatively, or in addition, the or each second control signal may be computer readable signal(s).

The output module may generate said one or more control signals based on power estimation in a respective bandpass filter of the filter module, although alternatives to the use of power estimation are possible.

The apparatus may further comprise a decomposition module for generating said neuron discharge patterns from said electromyography signals. The decomposition module may extract discharge timing of motor neurons, enabling decoding of instructions from individual motor neurons to drive specific outputs at specific times.

The apparatus may further comprise a neural interface for obtaining said electromyography signals. The electromyography signals may, for example, be obtained from a nervous system (e.g. a human nervous system). The electromyography signals may be received in real time (or near real time). In some example embodiments, the neural interface may comprise: an electrode array for measuring surface electrical signals; and a signal conditioning module for generating the electromyography signals from the surface electrical signals.

The apparatus may further comprise a configuration module for generating said bandpass filter configuration parameters. The configuration module may comprise an input for receiving calibration data (for example from a calibration module, as described below).

The apparatus may further comprise a calibration module. The calibration module may comprise: a first module for dividing neuron discharge patterns obtained from electromyography signals into first and second subsets; a second module for determining shared frequencies (e.g. in the form of intramuscular coherence) between said first and second subsets; and a third module for setting bandpass filter parameters for separating neuron discharge patterns obtained from electromyography signals into one or more frequency band signals based on said determined shared frequencies. The second module may further comprise: a filtering module for computing filtered first and second cumulative spike trains for the first and second subsets of discharge patterns respectively; and a power estimation module for estimating power levels of each filtered first and second cumulative spike train.

In another embodiment, there is provided an apparatus (e.g. a calibration module) comprising: a first module for dividing neuron discharge patterns obtained from electromyography signals into first and second subsets (e.g. random subsets); a second module for determining shared frequencies (e.g. in the form of intramuscular coherence) between said first and second subsets; and a third module for setting bandpass filter parameters for separating neuron discharge patterns obtained from electromyography signals into one or more frequency band signals based on said determined shared frequencies. The second module may further comprise: a filtering module for computing filtered first and second cumulative spike trains for the first and second subsets of discharge patterns respectively; and a power estimation module for estimating power levels of each filtered first and second cumulative spike train.

In a further embodiment, there is provided a method comprising: separating neuron discharge patterns obtained from electromyography signals into one or more frequency band signals based on bandpass filter configuration parameters; and converting one or more of said one or more frequency band signals into a one or more control signals, wherein said one or more control signals include one or more first control signals, wherein the or each first control signal is an independent augmented control signal.

The one or more control signals may further comprise one or more second control signals, wherein the or each second control signal is independent of said first control signal. The second control signal(s) may be muscle control signal(s) or some other control signal associated with the natural function of the muscle (e.g. a natural force control signal). The or each second control signal may be provided to one or more external electrical/electro-mechanical apparatus (e.g. a prosthetic limb). Alternatively, or in addition, the or each second control signal may be computer readable signal(s).

The output module may generate said one or more control signals based on power estimation in a respective bandpass filter of the filter module.

The method may further comprise: dividing neuron discharge patterns obtained from electromyography signals into first and second subsets; determining shared frequencies (e.g. in the form of intramuscular coherence) between said first and second subsets; and setting bandpass filter parameters for separating neuron discharge patterns obtained from electromyography signals into one or more frequency band signals based on said determined shared frequencies.

In yet another embodiment, there is provided a method comprising: dividing neuron discharge patterns obtained from electromyography signals into first and second subsets (e.g. two random subsets); determining shared frequencies (e.g. in the form of intramuscular coherence) between said first and second subsets; and setting bandpass filter parameters for separating neuron discharge patterns obtained from electromyography signals into one or more frequency band signals based on said determined shared frequencies.

The embodiments described herein may be used for many purposes, for example: in therapy; in rehabilitation or assistive devices; in controlling prosthetics; or in human-machine interaction (such as virtual reality/augmented reality applications, consumer electronics, computing applications, gaming etc.).

In another embodiment, there is provided a computer program comprising instructions for causing an apparatus to perform (at least) any of the methods set out above.

In yet another embodiment, there is provided a computer-readable medium (such as a non-transitory computer readably medium) comprising program instructions stored thereon for performing (at least) any of the methods set out above.

### BRIEF DESCRIPTION OF THE DRAWINGS

Example embodiments will now be described, by way of example only, with reference to the following schematic drawings, in which:

FIG. 1 is a block diagram of a neural interface;

FIG. 2 is a block diagram of a system showing an example use of a decomposition module of the neural interface of FIG. 1;

FIG. 3 is a block diagram of a system in accordance with an example embodiment;

FIG. 4 is a flow chart showing an algorithm in accordance with an example embodiment;

FIGS. 5 and 6 shows filter arrangements in accordance with example embodiments;

FIG. 7 is a block diagram of a system in accordance with an example embodiment;

FIG. 8 is a flow chart showing an algorithm in accordance with an example embodiment;

FIG. 9 is a flow chart showing an algorithm in accordance with an example embodiment; and

FIG. 10 is a block diagram of a system in accordance with an example embodiment.

### DETAILED DESCRIPTION

In the description and drawings, like reference numerals refer to like elements throughout.

Human-machine-interfaces (HMI) present a prospect for improving, through advancement of scientific knowledge and development of rehabilitation devices, the lives of millions of people who suffer from various neuromuscular disorders, paralysis and amputations worldwide. In addition, HMI have far reaching applications beyond such applications. With consumer electronics and Internet-of-Things (IoT) devices becoming integral parts of human daily life, HMI also presents the opportunity for humans to control their environment through their thoughts. HMI systems typically include a neural interface which taps into motor and sensory pathways to observe (i.e. record) and modulate (i.e. stimulate) the activity of the nervous system. Robust, reliable, accurate, adaptive, high-information throughput and easy-to-deploy neural interface technologies contribute to realising the potential and fulfilling the promises of future HMI. In this document, the inventors disclose such a device for neural interfaces suitable for a range of applications including research, clinical user consumer electronics use, virtual reality (VR) or augmented reality (AR) application, gaming etc.

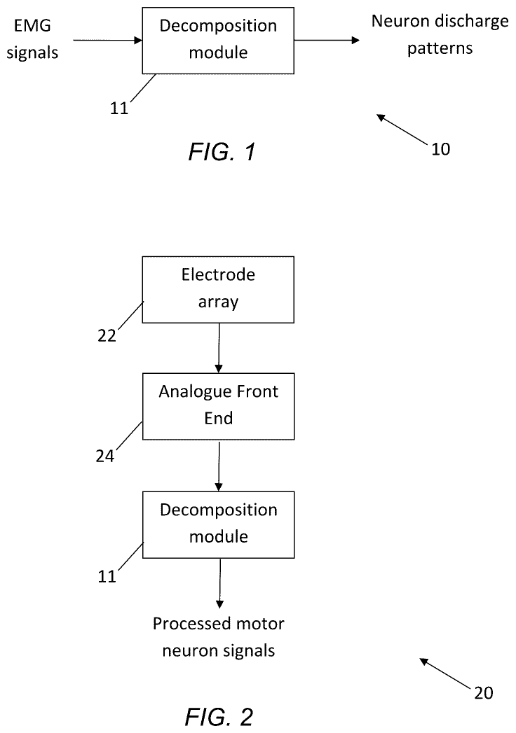

FIG. 1 is a block diagram of a neural interface, indicated generally by the reference numeral 10. The neural interface 10 comprises a decomposition module 11 (e.g. a real-time or near real-time decomposition module). The decomposition module 11 receives electromyography (EMG) signals at an input and provides estimates of neuron discharge patterns (e.g. motor neuron signals) at an output.

A number of embodiments described herein refer to “real-time” processing (such as real-time decomposition). In the context of this description, the term “real-time” is intended to cover “near real-time” processing in which a small time delay may occur (e.g. a transmission or processing delay). In such near real-time scenarios, no delay of significance to a user occurs.

FIG. 2 is a block diagram of a system, indicated generally by the reference numeral 20, demonstrating an example use of the decomposition module 11. The system 20 comprises the decomposition module 11 described above and further comprises an electrode array 22 and an analogue front end 24.

The electrode array 22 obtains EMG signals (e.g. surface EMG signals). The EMG signals, as detected by the electrode array, relate to the electrical activity of muscles and are the result of commands (i.e. electrical activity) sent from the brain via a network of specialised cells (i.e. neurons) running through spinal cord and connecting to the muscle fibres. The EMG signals can be modelled mathematically as the summed electrical activity of muscle fibres (within a muscle tissue) caused by the activity of motor neurons at the spinal cord level.

The obtained EMG signals are provided to the analogue front-end (AFE) 24 of the system 20. The AFE 24 includes an analogue-to-digital converter (ADC) and is used to amplify, filter and digitise the EMG signals provided by the electrode array 22. The output of the AFE 24 is provided to the decomposition module 11. The decomposition module 11 generates processed motor neuron signals, as discussed above.

Thus, the decomposition module 11 can be used to decipher motor neuron activity within the output layers of spinal cord from the EMG signals obtained by the electrode array 22. The decomposition module 11 may therefore receive the electromyography (EMG) signal and output the time occurrence of each motor neuron activity (i.e. spike discharges) constituting the EMG signal.

A challenge in human-machine interfacing (HMI) is the expansion of human capabilities by providing novel ways to interact with an environment. Real motor augmentation through supernumerary artificial limbs may provide users with new means of interaction with the environment through artificial actuators controlled using spare biological signals. However, in most cases, human motor augmentation is achieved at the expense of limiting already existing motor functions, e.g., by making users learn to control endogenous bio-signals that would otherwise be used for the movement of existing parts of the body. Identifying biological signals that can be reliably decoded and volitionally controlled independently from natural motor function is critical to achieve real human motor augmentation.

Neural or muscular signals may be used to implement human-machine interfaces. As described above, surface electromyography (sEMG) represents a non-invasive, direct and simple source of information tightly linked to the intent to move natural limbs. Due to biomechanical constraints, the effective frequency band within which common inputs to motor neurons (MNs) translate into movements is rather narrow and concentrated at frequencies <7 Hz. EMG information contained at higher frequencies, such as the cortical beta band (13-30 Hz), is known to be caused, at least in part, by projections from the motor cortex. As described in detail below, if information contained at higher frequencies observed in the muscles can be uncorrelated from motor function, they can be used to effectively expand the degrees-of-freedom of humans to control artificial limbs by enabling a new control channel independent from the existing natural ones. In fact, in principle this approach could lead to duplicating the degrees of freedom of muscles from which sources of information independent of natural motor control (e.g. high frequency sources outside the bandwidth of normal motor control) are extracted.

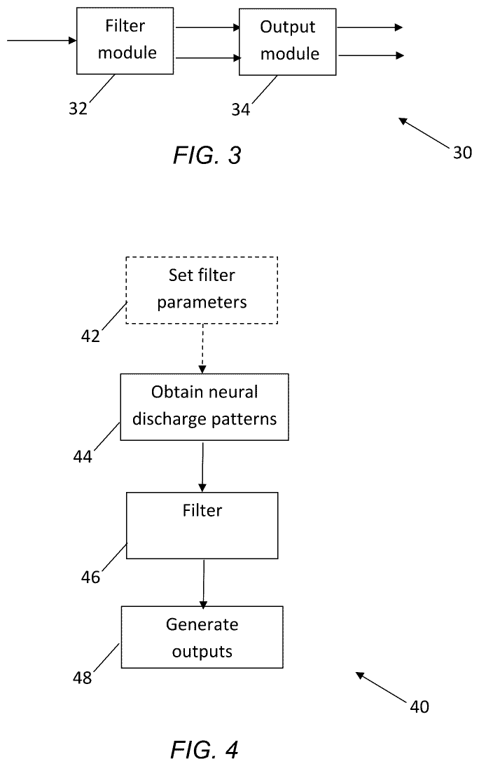

FIG. 3 is a block diagram of a system, indicated generally by the reference numeral 30, in accordance with an example embodiment. The system 30 comprises a filter module 32 and an output module 34.

The filter module 32 receives neuron discharge patterns, such as the output of the decomposition module 11 discussed above with reference to the systems 10 and 20.

The filter module 32 separates the neuron discharge patterns obtained from the electromyography signals into one or more frequency band signals based on bandpass filter configuration parameters.

The output module 34 converts one or more of said one or more frequency band signals into a one or more control signals (two are shown in the system 30), wherein said one or more control signals include one or more first control signals, wherein the or each control signal is an independent augmented control signal.

Using the system 30, spectral components within particular frequency ranges in the common inputs of the received motor neuron signals pool can be partly decoupled (e.g. beta-band signals can be separated from the low-frequency components) and therefore used to control additional degrees-of-freedom concurrently with the motor control of natural limbs.

Thus, the system 30 utilises the time occurrence of each motor neuron activity, in order to extract control signals independent of the intended action of the muscle. In this way, the outputs of the output module 34 are capable of providing control signals unrelated to (i.e. independent of) the natural behaviour of the muscle from which the EMG signals are being recorded. Furthermore, these independent control signals can be modulated voluntarily and augment human capabilities.

FIG. 4 is a flow chart showing an algorithm, indicated generally by the reference numeral 40, in accordance with an example embodiment.

The algorithm 40 starts at operation 42, where filter parameters (for example parameters of the filter module 32) are set. As discussed in detail below, the filter parameters may be configuration parameters that are set by a calibration module and may take the form of bandpass filter settings.

At operation 44, neural discharge patterns are obtained (for example as output by the decomposition module 11 described above).

At operation 46, the neural discharge patterns obtained in the operation 44 are filtered on the basis of the filter parameters set in the operation 42. In the operation 46, the neuron discharge patterns obtained in the operation 44 are separated into one or more frequency band signals, for example based on bandpass filter configuration parameters.

At operation 48, an output is generated (e.g. by the output module 34) in the form of one or more control signals that are converted from the one or more frequency band signals. The one or more control signals include one or more first control signals, wherein the or each first control signal is an independent augmented control signal, as discussed in detail below.

By way of example, one control signal may be a muscle control signal and may, for example, be provided to an external electrical/electro-mechanical apparatus (such as a prosthetic limb). Another control signal may entirely independent of the first control signal and may be used for any purposes—such as a control signal for operating an apparatus or a software module.

It should be noted that it is not essential that one of the first and second control signals is a muscle control signal. Indeed, both control signals may be provided for other purposes. It is also not essential that two control signals be provided—one or more signals may be provided in example embodiments.

In the algorithm 40, the operation 42 may be a one-time operation, or may be performed less often than the operations 44 to 48, such that the algorithm 40 may return to the operation 44 after the operation 48 has been carried out. Moreover, the operation 42 may be omitted entirely in some example embodiments (e.g. if filter parameters are pre-defined or set in some other way).

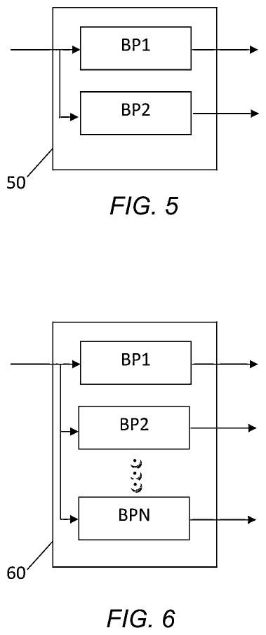

FIG. 5 shows a filter arrangement 50 in accordance with an example embodiment. The filter arrangement 50 includes a first bandpass filter BP1 and a second bandpass filter BP2 that are centred at different frequencies (which frequencies may be set in the operation 42 described above). The filter arrangement 50 can therefore be used to implement the filter module 32 for separating neuron discharge patterns obtained from electromyography signals into one or more frequency band signals based on bandpass filter configuration parameters.

Many variants of the filter arrangement 50 are possible. For example, FIG. 6 shows a filter arrangement 60 in accordance with an example embodiment comprising a plurality of bandpass filters (filter BP1, BP2 and BPN are shown by way of example). In principle, the filter module 32 can be used to separate neuron discharge patterns into any number of frequency bands.

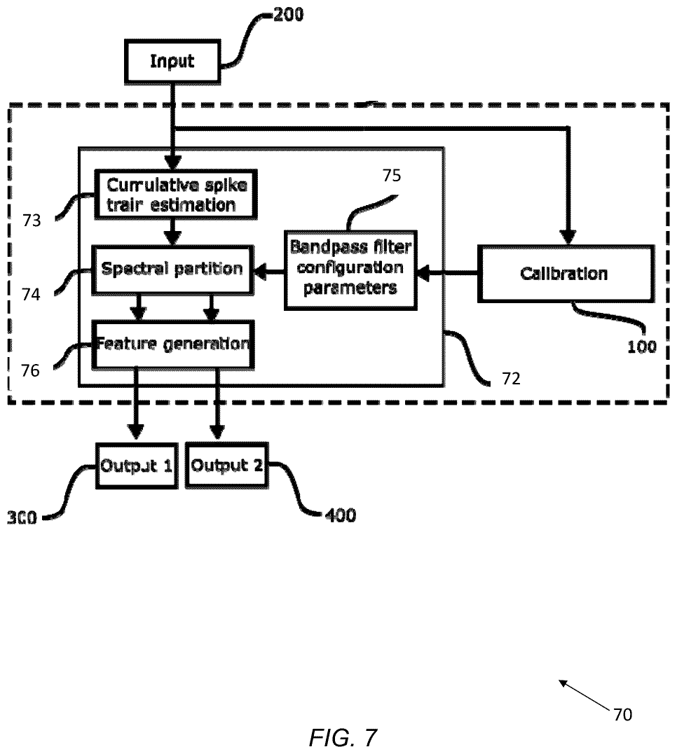

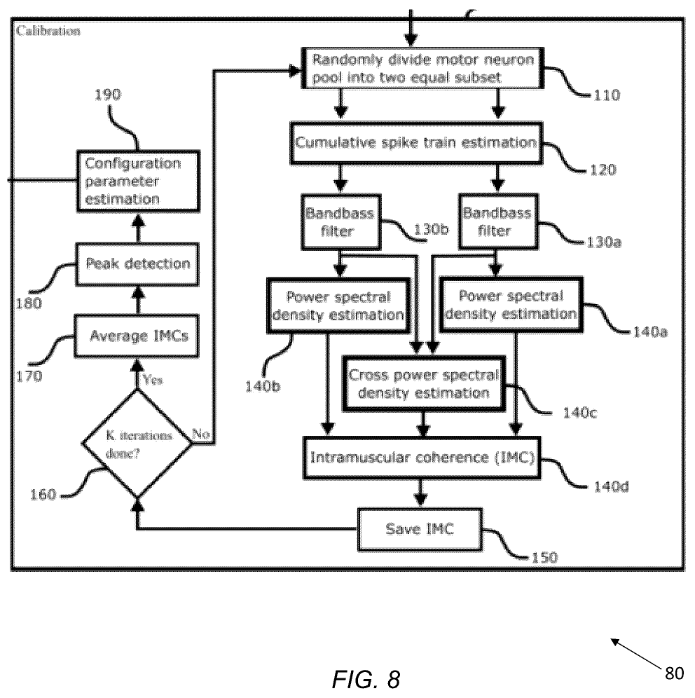

FIG. 7 is a block diagram of a system, indicated generally by the reference numeral 70, in accordance with an example embodiment.

The system 70 comprises a first module 72 and a second module 100. The first module 72 generates a first output 300 and an independent second output 400. For example, the first output 300 may provide control signals associated with natural force control (e.g. an output to control a limb, such as a prosthetic limb) and the second output may provide an output entirely independent of the force control signal. The first module 72 is an example implementation of the system 30 described above.

The second module 100 computes the configuration parameters of the bandpass filters used in the spectral partition module.

The system 70 receives an input 200 that is the time occurrence of each motor unit activity extracted from the EMG signal. The input 200 may be the output of the decomposition module 11 described above.

FIG. 8 is a flow chart, indicated generally by the reference numeral 80, showing an algorithm in accordance with an example embodiment. The algorithm 80 may be implemented by the calibration module 100. However, it should be noted that the calibration module 100 may be implemented in other ways and may be omitted altogether. For example, the bandpass filters of the system 70 may be hard coded to particular frequencies or those frequencies may be set in other ways (e.g. they may be user-configurable parameters).

In operation 110, the set of motor neurons input (the input 200—such as the output of the decomposition module 11) are divided into two sets of equal elements. Motor neurons for each set may be selected at random. This is followed, in operation 120, by computation of the cumulative spike train for each set—i.e. a single discharge pattern representing all time occurrences of all motor neurons within the sets as divided in the operation 110.

Next, bandpass filtering (130a and 130b) of each cumulative spike train is conducted (for example between 13-30 Hz, to extract the beta band of the signal). The bandpass filtering is followed by power spectral density estimations (operation 140a and 140b) and cross power spectral density (operation 140c).

The outputs of operations 140a, mob and 140c are used to compute intramuscular coherence (IMC) mod to quantify common inputs to the motor neuron pool in the different frequency bands. This is then repeated K times (operation 160) and the resulting IMCs are averaged (operation 170). The peak of the computed average IMC is then found (operation 180). This peak indicates the frequency f_{imc} at which there is strong common signal to all motor neurons. This is followed by configuration parameter estimation step 190 where the bandpass filter calibration <r parameters are set between f_{imc}−x and f_{imc}+x. The value x may be 2.5 Hz by default, however, could be set to any other value (some embodiments require that f_{imc}−x and f_{imc}+x are within beta band, but this is not essential to all example embodiments). Moreover, the two bandpass filters may be of different sizes (i.e. the size of the “x” referred to above may be different in different bandpass filters) This process can also be automated for optimal selection of the band.

With the configuration parameters set (either using the algorithm 80 or in some other way), the system 70 performs real-time computation and extraction of the features. First, the cumulative spike train—i.e. a single discharge pattern representing all time occurrences of all motor neurons—is computed using spike train estimation module 73. This is followed by spectral partition (using spectral partition module 74) during which the estimated cumulative spike train is broken down into low-frequency and high-frequency components through filtering (e.g. based on parameters stored within bandpass filter configuration parameters module 75). In one example embodiment, the low frequency component refers to the portion of the signal between 1 and 1+2x Hz, while the high-frequency components refer to the portion of the signal between f_{imc}−x and f_{imc}+x (see the description of calibration above). This is followed by feature generation during which the power of each filtered cumulative spike train is estimated (using feature generation module 76). As noted above, the output signal 400 is a control signal that is unrelated (i.e. independent) to the natural behaviour of the muscle, while 300 may be associated with the natural behaviour (i.e. force output) of the muscle. Both 300 and 400 may each comprise multiple outputs/features.

The feature generation module 76 described above uses power estimation to determine features within the spectrally partitioned signals. This is not essential to all example embodiments. Power extraction, as described above, is one way in which this can be implemented, but alternatives are possible.

Using the systems 30 and 70 described above, spectral components within particular frequency ranges (such as the beta range, gamma range etc.) in the common inputs to received motor neuron signals pool can be partly decoupled from other frequencies (e.g. low-frequency components) and therefore used to control additional degrees-of-freedom concurrently with the motor control of natural limbs.

The algorithm 80 described above provides a method for setting filter parameters. This is not essential to all embodiments; many alternative algorithms are possible.

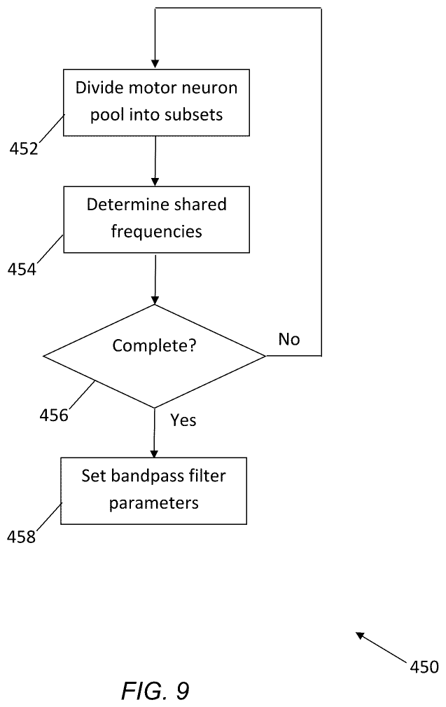

FIG. 9 is a flow chart showing an algorithm, indicated generally by the reference numeral 450, in accordance with an example embodiment. The algorithm 450 is a variant of the algorithm 80 described above.

The algorithm 450 starts at operation 452, where neuron discharge patterns obtained from electromyography signals are divided (e.g. randomly) into first and second subsets. The operation 450 may therefore be the same as the operation 110 described above.

Next, at operation 454, shared frequencies between said first and second subsets are determined. As described above with reference to the algorithm 80, the shared frequencies may be in the form of intramuscular coherence (IMC). Such shared frequencies may be determined in many ways—the specific example described with reference to the algorithm 80 is one of many examples.

At operation 456, a determination is made regarding whether sufficient data has been processed. If so, the algorithm 450 proceeds to operation 458, otherwise, the algorithm returns to operation 452 and further motor neuron data is processed. The operation 456 may, for example, be the operation 160 of the algorithm 80.

Finally, at operation 458, bandpass filter parameters are set. The operation 458 may include some or all of the operations 170, 180 and 190 described above.

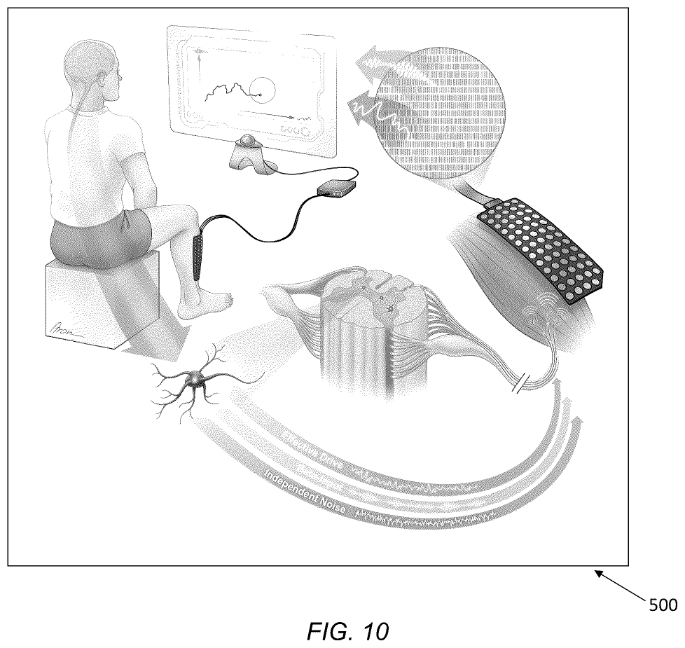

FIG. 10 is a block diagram of a system, indicated generally by the reference numeral 500, in accordance with an example embodiment, that was developed to test the principles described above.

In the system 500, the spinal motor neuron (MN) pool projecting to the tibialis anterior muscle of the right leg received common neural projections in a broad frequency range. These projections were demodulated by the MN pool to produce an output neural signal that includes low frequency components driving movements (effective drive to the muscle; <7 Hz). In addition, MNs send common projections to the muscle in the beta band (˜20 Hz) and each neuron received an additional input, referred to as independent noise. Spiking activity of MNs was decomposed in real-time from high-density surface EMG. Decomposed spiking activity was used to retrieve information about the effective drive and beta projections.

The system 500 used a real-time EMG decomposition technique to extract the firing patterns of pools of MNs projecting to a single muscle to directly decode the neural input the muscle receives. These firing patterns allowed information about the driving of the muscle (components at low frequencies causing muscle contraction and movements) from common projections to the MN pool in the beta band (˜20 Hz) that are not directly translated into force because of their high frequency relative to the musculoskeletal bandwidth. With this, it was confirmed that subjects could independently control components in the neural projections to muscles that do not directly determine force and that are therefore not directly associated with the natural control of movements.

The principles described herein seek to enable the augmentation of humans with supernumerary degrees of freedom that can be controlled as intuitively and independently as our natural ones. Such technologies may extend human motor capacities beyond natural anatomical boundaries.

The principles described herein enable control commands to be provided to any device, independent of an underlying action of the controlling muscle, thereby enabling the device to be controlled independently without hindering the functionality of the controlling muscle.

Embodiments of the presented invention may be implemented in software, hardware, application logic or a combination of software, hardware and application logic. The software, application logic and/or hardware may reside on memory, or any computer media.

If desired, the different functions discussed herein may be performed in a different order and/or concurrently with each other. Furthermore, if desired, one or more of the above-described functions may be optional or may be combined.

It will be appreciated that the above described example embodiments are purely illustrative and are not limiting on the scope of the invention. Other variations and modifications will be apparent to persons skilled in the art upon reading the present specification.

Moreover, the disclosure of the present application should be understood to include any novel features or any novel combination of features either explicitly or implicitly disclosed herein or any generalization thereof and during the prosecution of the present application or of any application derived therefrom, new claims may be formulated to cover any such features and/or combination of such features.

Although various aspects of the invention are set out in the independent claims, other aspects of the invention comprise other combinations of features from the described example embodiments and/or the dependent claims with the features of the independent claims, and not solely the combinations explicitly set out in the claims.

1. An apparatus comprising:
a filter module for separating neuron discharge patterns obtained from electromyography signals into one or more frequency band signals based on bandpass filter configuration parameters; and
an output module for converting one or more of said one or more frequency band signals into a one or more control signals, wherein said one or more control signals include one or more first control signals, wherein the or each first control signal is an independent augmented control signal.

2. An apparatus as claimed in claim 1, wherein the one or more control signals further comprise one or more second control signals, wherein the or each second control signal is a muscle control signal independent of said first control signal.

3. An apparatus as claimed in claim 2, wherein the or each second control signals are provided to one or more external electrical/electro-mechanical apparatus.

4. An apparatus as claimed in claim 1, wherein the output module generates said one or more control signals based on power estimation in a respective bandpass filter of the filter module.

5. An apparatus as claimed in claim 1, further comprising a configuration module for generating said bandpass filter configuration parameters.

6. An apparatus as claimed in claim 5, wherein said configuration module comprising an input for receiving calibration data.

7. An apparatus as claimed in claim 1, further comprising a decomposition module for generating said neuron discharge patterns from said electromyography signals.

8. An apparatus as claimed in claim 7, wherein the decomposition module extracts discharge timing of motor neurons, enabling decoding of instructions from individual motor neurons to drive specific outputs at specific times.

9. An apparatus as claimed in claim 1, further comprising a neural interface for obtaining said electromyography signals.

10. An apparatus as claimed in claim 9, wherein said neural interface comprises:
an electrode array for measuring surface electrical signals; and
a signal conditioning module for generating the electromyography signals from the surface electrical signals.

11. An apparatus as claimed in claim 1, wherein the apparatus is a human-machine interface.

12. An apparatus comprising:
a first module for dividing neuron discharge patterns obtained from electromyography signals into first and second subsets;
a second module for determining shared frequencies between said first and second subsets; and
a third module for setting bandpass filter parameters for separating neuron discharge patterns obtained from electromyography signals into one or more frequency band signals based on said determined shared frequencies.

13. An apparatus as claimed in claim 12, wherein the second module comprises:
a filtering module for computing filtered first and second cumulative spike trains for the first and second subsets of discharge patterns respectively; and
a power estimation module for estimating power levels of each filtered first and second cumulative spike train.

14. A system comprising an apparatus as claimed in claim 1.

15. A method comprising:
separating neuron discharge patterns obtained from electromyography signals into one or more frequency band signals based on bandpass filter configuration parameters; and
converting one or more of said one or more frequency band signals into a one or more control signals, wherein said one or more control signals include one or more first control signals, wherein the or each first control signal is an independent augmented control signal.

16. A method comprising:
dividing neuron discharge patterns obtained from electromyography signals into first and second subsets;
determining shared frequencies between said first and second subsets; and
setting bandpass filter parameters for separating neuron discharge patterns obtained from electromyography signals into one or more frequency band signals based on said determined shared frequencies.

17. The apparatus of claim 1, for use in therapy.

18. The apparatus of claim 1, for use in rehabilitation or assistive devices.

19. The apparatus of claim 1, for use in controlling prosthetics.

20. The apparatus of claim 1, for use in human-machine interaction.
